(19) United States
(12) Patent Application Publication
(10) Pub. No.: US 2006/0149249 A1
(43) Pub. Date: Jul. 6, 2006

(54) Osteosynthesis plate for the osteosynthesis of small neighbouring bones

(76) Inventors: Christophe Mathoulin, Paris (FR); Jean-Jacques Martin, Bourg En Bresse (FR)

Correspondence Address: YOUNG & THOMPSON, 745 SOUTH 23RD STREET, 2ND FLOOR, ARLINGTON, VA, 22202, US

(21) Appl. No.: 10/538,809

(22) PCT Filed: Oct. 22, 2003
(86) PCT No.: PCT/FR03/03129

§ 371 Date: Dec. 23, 2005

(30) Foreign Application Priority Data
Dec. 11, 2002 (FR) 02/15614

(51) Int. Cl. A61F 2/30

(52) U.S. Cl. 606/69

(57) ABSTRACT

The invention relates to an osteosynthesis plate for the osteosynthesis of small neighbouring bones. The inventive osteosynthesis plate (1) is dimensioned such that it can be positioned above the bones to be treated without resting on the larger neighbouring bones. According to the invention, the osteosynthesis plate comprises lateral holes (16) for the insertion of screws (6) which are used to fix same to the bones (2 to 5). The plate is characterised in that: (i) it comprises a flat face (10) which is intended to come into contact with the bones to be treated; and (ii) it comprises holes (16) for receiving screws (6), which have axes that are inclined in relation to the aforementioned flat face (10) and which are designed such that the inserted screws diverge towards the outside of the plate (1).

### FIELD OF THE INVENTION

The present invention refers to an osteosynthesis plate for the osteosynthesis of small neighbouring bones, in particular wrist bones. The invention also refers to a set of instruments for installing such plate.

### BACKGROUND OF THE INVENTION

It is necessary in some cases to perform an osteosynthesis of small neighbouring bones. In particular, when dealing with wrist bones, there may be provided an osteosynthesis of the bone capitatum, of the semi-lunar bone, of the cuneiform bone and of the unciform bone.

To conduct such osteosynthesis, osteosynthesis plates may be used conventionally, resting on both sides of the carpus, in particular on the radius on the one hand and on one or several metacarpal bones on the other hand. The major shortcoming of such plates is to block the wrist joint.

There is provided moreover an osteosynthesis plate, dimensioned such that it can be positioned above the small bones to be assembled, hence without resting on larger neighbouring bones, in particular the radius and one or several metacarpal bones in the case of an osteosynthesis of wrist bones. Such plate is annular in shape, with a wide central opening, is conical in shape as seen transversally, which delineates a lower resting rim against the bones to be treated, and exhibits eight lateral holes for inserting screws into said bones.

The major shortcoming of such plate lies in that it is relatively difficult to implant while enabling the insertion of screws not in the bones to be treated, but between two of such bones, which may prove detrimental later on.

The present invention intends to remedy such shortcomings.

### SUMMARY OF THE INVENTION

The plate in question is of the type having dimensions such that it may be placed above the bones to be treated, without resting on larger neighbouring bones, and comprising lateral holes for inserting screws to fix the latter to such bones.

According to the invention, the plate comprises a face intended to come into contact with the bones to be treated which is flat, and comprises holes for receiving screws having axes that are tilted in relation to said flat face, such holes being designed such that the screws, once inserted into said holes, diverge towards the outside of the plate.

The plate according to the invention, because of the flatness of the face thereof intended to come into contact with the bones to be treated, only involves countersinking the bones to be treated, which may be provided by dint of a “countersink”-type reamer, and offers a perfect resting grip against the bones to be treated. Such plate is much easier to be implanted than the existing plate. The tilted holes enable to orient the screw towards the bones wherein said screws should be implanted, notwithstanding the flat face of the plate.

The plate may be circular in shape, and said reamer may then be hollowed and liable to be engaged slidingly, but with a tight fit, on a positioning spindle.

Said spindle thus enables to guide the rotation of the reamer and to provide a recess adapted precisely to the shape of the plate.

The face of the plate opposite to that coming into contact with the bones to be treated may exhibit a recess enabling to lower the heads of the screws with respect to the plate in implanting position thereof.

Such screw heads do not protrude significantly beyond said face of the plate and cannot damage the neighbouring tissues.

Such recess may in particular occupy the major portion of said face of the plate opposite to that coming into contact with the bones to be treated, and be in the form of a hollow spherical cap.

The recess thus shaped enables moreover to delineate tilted zones with respect to the general plane of the plate, through which the screw holes may be laid out, perpendicular to said zones.

Preferably, at least one screw hole is in the form of a hollow spherical section, and the head of at least one screw exhibits a side wall in the form of matching spherical section, these respective shapes of the hole and of the screw head enabling multidirectional orientation of the screw with respect to the plate.

Such multidirectional orientation enables to adapt the insertion direction of the screw, if rendered necessary in relation to the relative positions of the plate and of a bone to be treated.

Preferably, the plate comprises a number of screw holes equal to the number of bones to be treated, or close to such number, in particular four holes to perform, when dealing with wrist bones, osteosynthesis of the bone capitatum, of the semi-lunar bone, of the cuneiform bone and of the unciform bone. Practically, the plate will comprise generally three to five holes.

This number of screw holes just necessary contributes to reducing the risk of insertion of a screw between two bones.

According to another aspect of the invention, the plate comprises a central hole of diameter adjusted to that of a positioning spindle, enabling the sliding engagement of the plate on said spindle, and a mark situated at the periphery thereof.

The positioning spindle is thus inserted in one of the bones to be treated, or between the bones to be treated, then the plate is engaged on said spindle and is oriented angularly so that the lateral screw holes included in such plate are facing the bones to be treated, the adequate angular position of the plate being ensured by dint of said mark. The screws fastening the plate to the bones to be treated are then inserted.

The plate according to the invention, thanks to its accurate positioning made possible by said spindle and said mark, eliminates, or at least reduces quite considerably, the risk of insertion of a screw between two bones.

The set of instruments for the insertion of the plate may comprise, in addition to said reamer, a dummy of the plate, i.e. a test piece identical in shape to that of the plate, provided with a mark identical to that of the plate.

Such dummy piece is placed in the position which ought to be occupied later on by the plate and is used for marking the adequate angular position which should be conferred to such plate so that the screw holes thereof are facing the different bones to be treated. Once this marking has been completed, a mark is placed on one of the bones to be treated, for example by means of an electric surgical knife, facing the mark of the dummy piece, then the latter is withdrawn. The adequate position of the plate will be easy to find at a later stage, simply by matching the mark on the plate with said other mark.

The dummy piece may advantageously be provided with a hole for receiving said positioning spindle, identical to that of the plate.

This dummy piece thus enables to determine the position of such spindle, which will allow placing the plate at a later stage.

The following description in conjunction with the appended drawings, given by way of non-limiting example, will enable to understand the invention better, the features exhibited as well as the advantages liable to be offered.

### BRIEF DESCRIPTION OF THE DRAWINGS

FIG. 1 is a perspective view representing the osteosynthesis plate according to the invention while performing osteosynthesis of the bone capitatum, of the semi-lunar bone, of the cuneiform bone and of the unciform bone; for simplification purposes of the drawing, such bones have been represented very schematically, untrue to reality, except as regards the contours thereof.

FIG. 2 is a top view illustrating such plate at enlarged scale.

FIG. 3 is a diametral sectional view along the line III-III of FIG. 2.

FIGS. 4 to 6 are top views of three plates similar to one another, but having different diameters and a different number of screw holes.

FIGS. 7 to 11 are views similar to FIG. 1, showing four successive steps of insertion of the plate.

### DESCRIPTION OF THE PREFERRED EMBIDOMENT

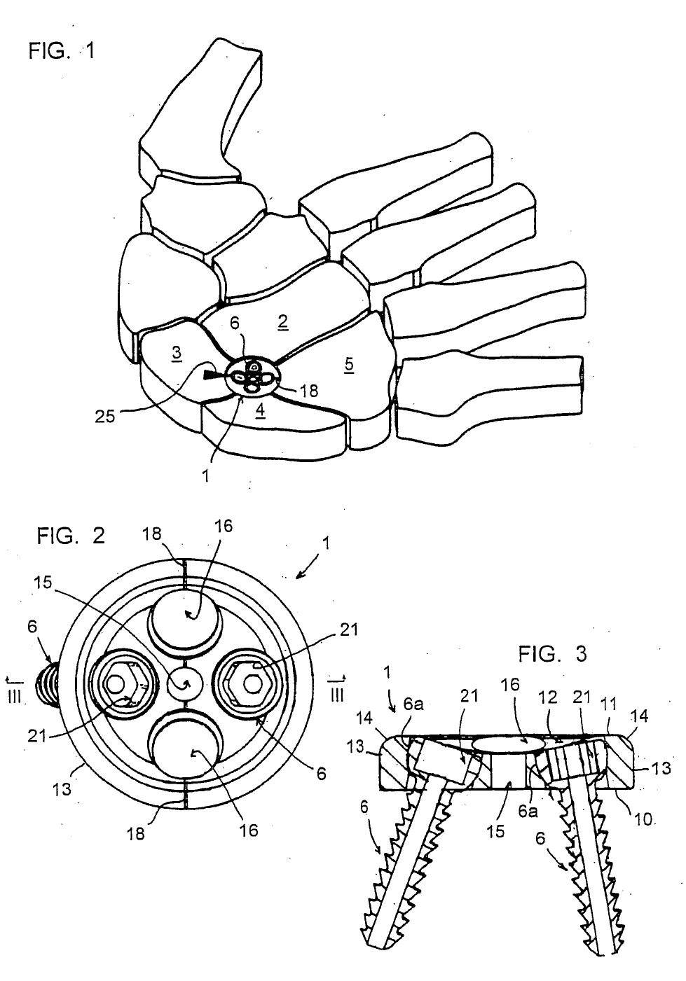

FIG. 1 shows an osteosynthesis plate 1 for the osteosynthesis of small neighbouring bones, in particular wrist bones. In the example represented, it relates to the osteosynthesis of the capitatum bone 2, of the semi-lunar bone, of the cuneiform bone and of the unciform bone. This plate 1 is fixed to said bones by means of screws 6 (only one is inserted on the plate 1, represented by FIG. 1).

Such plate 1 and such screws 6 of any appropriate material, in particular the titanium alloy known under the reference TA6V.

As shown more particularly on FIGS. 2 and 3, the plate 1 is cylindrical in shape, with a flat face 10 intended to come into contact with the bones to be treated, a face 11 opposite to the face 10, exhibiting a recess 12, and a peripheral face 13, connected to the face 11 by a non-sharp rounded angle 14.

The recess 12 is in the form of a hollow spherical cap and occupies the major portion of the face 11.

The plate 1 comprises a central hole 15, holes 16 for receiving the screws 6, and an engraved diametral line 18, extending over the face 11 and prolonging over the peripheral face 13.

The hole 15 is provided along an axis perpendicular to the general plane of the plate 1. It has a diameter adjusted to that of a positioning spindle 20, visible on FIG. 8, and enables the sliding engagement of the plate 1 on said spindle 20.

Each hole 16 is laid out substantially perpendicular to the area of the face wherein it emerges, so that, taking into account the curvature of the recess 12, the axis of each of such holes 16 is tilted towards the outside of the plate 1 according to an angle of the order of 10° with respect to the axis of the hole 15.

The zone of the plate 1 delineating each hole 16 is in the form of a hollow spherical section.

Each screw 6 is hollowed and self-drilling, and has a head exhibiting a side wall 6a in the form of a spherical section. Said side wall 6a has a shape matching that of the zones of the plate 1 delineating the holes 16, so that said wall 6a may rest on a surface in each hole 16, but with a possibility of multidirectional orientation of the screw 6 in the hole 16.

Each screw 6 exhibits moreover a proximal cavity 21, enabling handling thereof by means of an appropriate tool (not represented on the Figures), said cavity 21 having, in the example represented, hexagonal shape with rounded angles.

The diametral line 18 forms, at the periphery of the plate 1, a mark of angular positioning of this plate 1, as will be explained below.

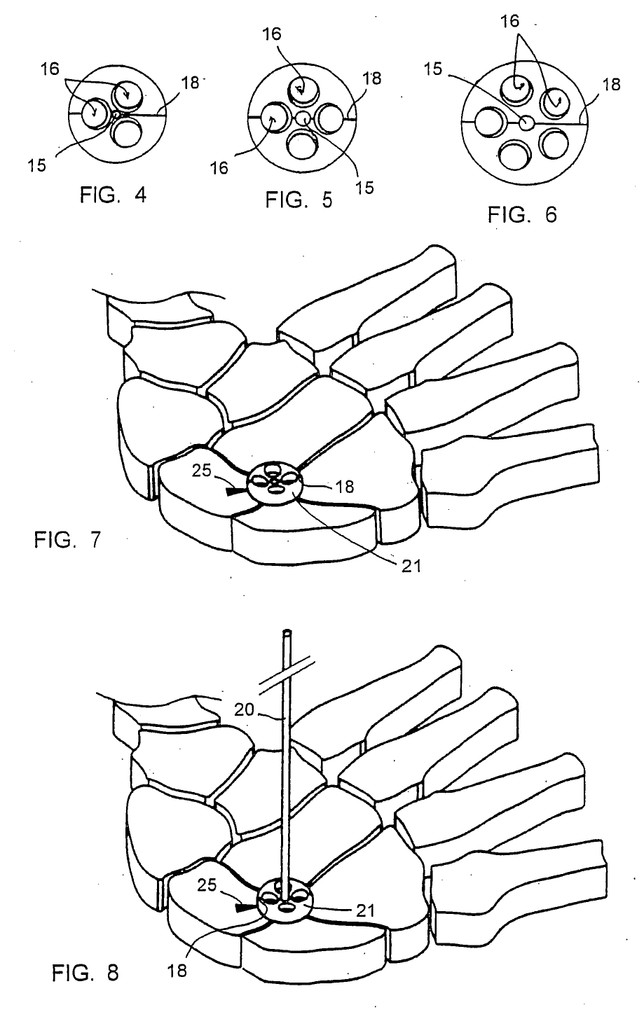

The plate 1 may comprise four holes 16 as shown on FIGS. 2, 3 and 5, or three or five holes 16 as shown respectively on FIGS. 4 and 6, according to the different types of bones liable to be treated by means of such a plate.

For information purposes, the following dimensions may be specified:

diameter of the plate 1 shown on FIGS. 2, 3 and 5: 14 mm;

thickness of this plate: 3 mm outside the recess 12; 2 mm at the level of the hole 15;

diameter of the hole: 1.9 mm;

distance between the centres of the spheres formed by said zones delineating the holes 16, from one hole 16 to an adjoining hole 16: 7 mm; amplitude of the possible clearance for the screws 6 in the holes 16: 10 degrees on both sides of the neutral position corresponding to a 10° tilting of the axis of such screws with respect to the axis of the hole 15.

The plate shown on FIG. 4 may have a diameter of 12.5 mm and a thickness of 3 mm outside the recess 12, and the plate shown on FIG. 6 may have a diameter of 16 mm and a thickness of 3 mm outside the recess 12.

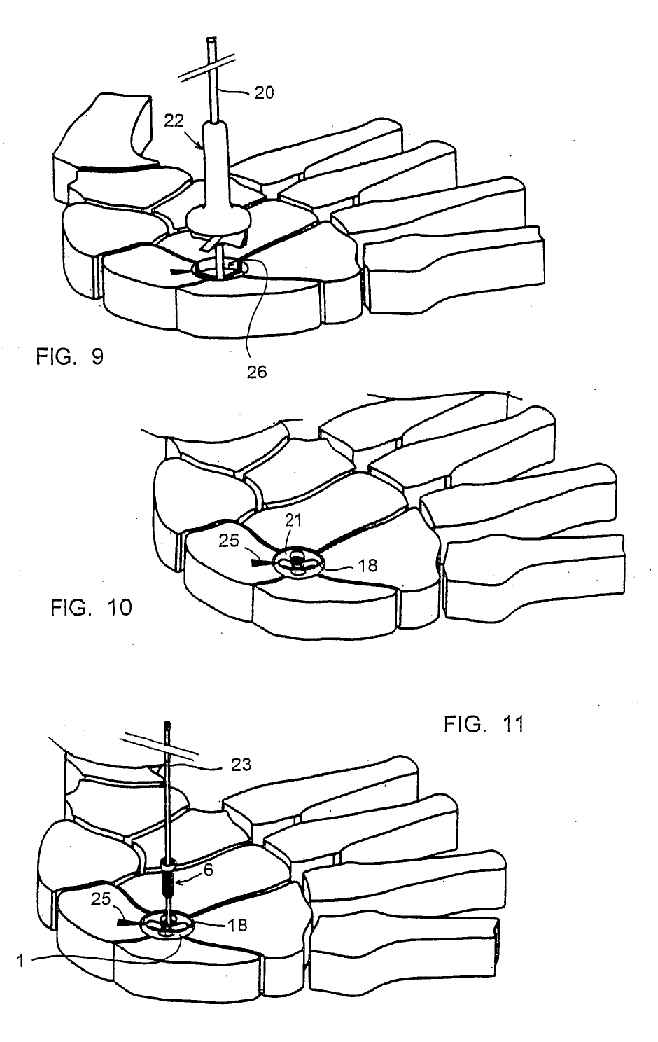

The material for the insertion of the plate 1 comprises, in addition to said positioning spindle 20, a dummy 21 of the plate 1, i.e. a test piece identical in shape to that of the plate 1, exhibiting holes 15 and 16 and a mark 18 identical to those of such plate, has a hollowed countersinking reamer 22, visible on FIG. 9, and spindles 23 for guiding the screws 6.

Practically, as represented on FIG. 7, the dummy piece 21 is positioned on the bones to be treated 2 to 5 and is oriented angularly so that the holes 16 contained therein match at best each bone 2 to 5. A mark 25 is then provided on one of the bones to be treated by means of an electric surgical knife, facing the mark 18 of the dummy piece 21.

The dummy piece 21 is held in this position, then the spindle 20 is placed through the hole 15 of such dummy piece, as represented on FIG. 8. The dummy piece 21 is then withdrawn by sliding along said spindle 20.

The countersinking reamer 22 is then inserted on the spindle 20 and is used for providing a countersink 26 coaxial to said spindle 20, as represented on FIG. 9.

The spindle 20 is withdrawn then the plate 1 is inserted in said countersink 26. The adequate position of this plate 1 determined by means of the dummy piece 21 can be found easily by matching the mark 18 contained in said plate 1 opposite the mark 25, as illustrated on FIG. 10.

The spindles 23 are then inserted one by one into the bones for guiding each screw 6 in the matching hole 16, as shown on FIG. 11.

The invention provides a plate for the osteosynthesis of small neighbouring bones, in particular bones of the carpus, whereof the implantation is made easier by the features of such plate and by the set of instruments 20 to 23 enabling the insertion thereof. Such plate eliminates moreover, or at least reduces very significantly, the risk of inserting a screw between two bones, thanks to accurate positioning of such plate made possible by said spindle 20 and said mark 18.

It goes without saying that the plate might exhibit any other form, without modifying the object of the present invention in any way.

It should be understood that the preceding description has only been given for exemplification purposes and that it does not limit the field of the invention without departing from the execution details described by any other equivalent.

1. Osteosynthesis plate for osteosynthesis of small neighbouring bones, in particular wrist bones, having dimensions such that it may be placed above the bones to be treated, without resting on larger neighbouring bones, and comprising lateral holes for inserting screws to fix the latter to such bones characterized in that it comprises a face intended to come into contact with the bones to be treated which is flat, and comprises holes for receiving screws having axes that are tilted in relation to said flat face, such holes being designed such that the screws, once inserted into said holes, diverge towards the outside of the plate.

2. Osteosynthesis plate according to claim 1, characterized in that it is circular in shape.

3. Osteosynthesis plate according to claim 1, characterized in that its face opposite to that coming into contact with the bones to be treated exhibits a recess enabling to lower the heads of the screws with respect to the plate in implanting position thereof.

4. Osteosynthesis plate according to claim 3, characterized in that the recess occupies the major portion of said face of the plate opposite to that coming into contact with the bones to be treated, and is in the form of a hollow spherical cap.

5. Osteosynthesis plate according to claim 1, characterized in that at least one screw hole is in the form of a hollow spherical section, and in that the head of at least one screw exhibits a side wall in the form of matching spherical section, these respective shapes of the hole and of the screw head enabling multidirectional orientation of the screw with respect to the plate.

6. Osteosynthesis plate according to claim 1, characterized in that it comprises a number of screw holes equal to the number of bones to be treated, or close to such number, in particular four holes to perform, when dealing with wrist bones, osteosynthesis of the bone capitatum, of the semi-lunar bone, of the cuneiform bone and of the unciform bone.

7. Osteosynthesis plate according to claim 1, characterized in that it comprises a central hole of diameter adjusted to that of a positioning spindle, enabling the sliding engagement of the plate on said spindle, and a mark situated at the periphery thereof.

8. Set of instruments for the insertion of the osteosynthesis plate according to claim 1, characterized in that it comprises a reamer.

9. Set of instruments according to claim 8, characterized in that it comprises a positioning spindle, enabling the sliding engagement of the plate on said spindle.

10. Set of instruments according to claim 8, characterized in that it comprises a dummy of the plate, i.e. a test piece identical in shape to that of the plate, provided with a mark identical to that of the plate.

11. Set of instruments according to claim 10, characterized in that the dummy provides with a hole identical to that of the plate, enabling to engage this dummy on said positioning spindle.

12. Set of instruments according to claim 9, characterized in that the reamer is hollowed and may be engaged by sliding, but with a tight fit, on said positioning spindle.

13. Set of instruments for the insertion of the osteosynthesis plate according to claim 2, characterized in that it comprises a reamer.

14. Set of instruments for the insertion of the osteosynthesis plate according to claim 3, characterized in that it comprises a reamer.

15. Set of instruments for the insertion of the osteosynthesis plate according to claim 4, characterized in that it comprises a reamer.

16. Set of instruments for the insertion of the osteosynthesis plate according to claim 5, characterized in that it comprises a reamer.

17. Set of instruments for the insertion of the osteosynthesis plate according to claim 6, characterized in that it comprises a reamer.

18. Set of instruments for the insertion of the osteosynthesis plate according to claim 7, characterized in that it comprises a reamer.
